(19) United States
(12) Patent Application Publication
(10) Pub. No.: US 2022/0225912 A1
(43) Pub. Date: Jul. 21, 2022

(54) FINGER NEEDLE PUNCTURE BLOOD COLLECTOR

(71) Applicant: INSTITUTE OF TROPICAL MEDICINE, Antwerpen (BE)

(72) Inventors: Alexander Nieuwborg, Wilrijk (BE); Frank Goethijn, Gent (BE)

(73) Assignee: INSTITUTE OF TROPICAL MEDICINE, Antwerpen (BE)

(21) Appl. No.: 17/614,756

(22) PCT Filed: Jun. 4, 2020
(86) PCT No.: PCT/EP2020/065461

§ 371 Date: Nov. 29, 2021

(30) Foreign Application Priority Data
Jun. 4, 2019 (BE) BE2019/5366

(51) Int. Cl. A61B 5/15; A61B 5/151

(52) U.S. Cl. CPC A61B 5/150022; A61B 5/150412; A61B 5/150748; A61B 5/150503; A61B 5/15117; A61B 5/150343; A61B 5/150977

(57) ABSTRACT

The current invention concerns an integrated medical device for a self-collection test, which allows for both puncturing and collecting a blood sample.

### TECHNICAL FIELD OF THE INVENTION

The current invention concerns an integrated medical device for a self-collection test, which allows for both puncturing and collecting a blood sample.

### BACKGROUND TO THE INVENTION

The current invention concerns a medical device with which the patient can, via puncturing of a finger, collect blood by him-/herself with a sample volume of approximately 500 μl. The result is an integrated system that allows for both puncturing and collection. In addition, the product is more hygienic, efficient, and user-friendly than current applications.

The self-collection test process can be divided into two phases: a preparatory phase and a needle puncturing and collection phase. The preparatory phase focuses on optimising blood flow to facilitate the easing of the steps to follow. In already existing self-collection tests, people are asked to first wash their hands with warm water to be followed by a hand massage. Afterwards, a finger to be selected must be disinfected with an alcohol wipe. A ring or index finger is preferred. The needle puncturing and collection phase commence when the finger is punctured by pricking a needle. This is followed by two choices. Either the released blood is collected with a capillary and it is then emptied into a tube that is sent later. Or the finger is simply allowed to leak into the same tube. This is referred to as “free fall”. Both methods will require massaging to get enough blood released. Once the tube is full, it can be sent. In conclusion, a plaster is placed around the finger to prevent further contamination.

Needle puncturing is an essential element of the entire blood collection process. The product presents in many different forms and sizes. We can identify two operating principles: reusable blood collection devices and disposable needle punctures. Disposable needle punctures are mainly used for medical applications for hygienic purposes. Renewable devices can frequently be used for glucose monitoring for diabetes. There frequently is a fixed part on which the disposable needle punctures could be placed. The focus of this invention is on disposable needle punctures, as these are better suited to one-off used self-collection tests. Relevant subgroups are the “twist off lancets” and “one step safety lancets”. The first principle is referred to as “twist off lancets”. These are needle punctures that have a plastic cover over their lancet. With a simple rotary movement of “twist off” it can be removed from the lancet and a puncture can be made. In practice, these needle punctures are used in a hospital environment. Current self-collection tests avoid these because the lancet is not covered after use, which increases the chance of puncture accidents. Twist off lancets do indeed frequently return in case of reusable needle punctures.

A second principle concerns the “one step safety lancets”. The product has a simple structure. The lancet comprises a lancet holder that holds the lancet. Both are linked to a spring holder by a spring. There is a knob that can be covered with the lid in front of the lancet. The parts referred to above are shielded with a casing. This in combination with the cover ensures a safely closed needle puncture that can be discarded without risk. The device can also be used for a single puncture and that offers additional safety. There are different versions of this system with an analogous function. The function of the one step safety lancet can be divided into 3 phases. During phase 1, the lancet is held by the lancet holder in 2 grooves. The spring that connects both has been tightened. The puncturing starts with phase 2, The finger is pressed against the knob. This, in turn, will push the lancet holder from the grooves. This is followed by the lancet, because of the tightened spring, being thrust forward and puncturing the finger. In phase 3, the needle puncture is lifted, and the knob returns to its original place. The spring will also quickly retract the lancet after the puncture to end up in a neutral resting position. This disables any further needle puncturing, by which accidents can be avoided successfully.

There is clearly still room for innovation with regard to these needle punctures, where existing shortcomings and problems (i.e. accidently activating the finger needle puncture, the lack of an integrated blood sample collection system, incorrect assembly in respect of the sample object) can be dealt with, avoided or reduced.

Our current invention concerns an elementary medical device that integrates puncturing and collection in a product that is manageable by a single person.

### SUMMARY OF THE INVENTION

A first embodiment of the invention concerns a device for the collection of a blood sample, comprising a holder for receiving the sample object, where the holder defines an opening for the attachment of a container, the container making a direct connection between the holder and the casing, and comprising a first opening and a closing mechanism for closing the first opening of the container, the casing comprises an opening, a perforation system and a spring, the perforation system being movable in the opening and the container between the first position and the second position by means of the spring, during which the perforation system, in the second position, finds itself at least partially outside of the container.

A second embodiment of the invention describes the device for the collecting of a blood sample according to the invention, in which the perforation system comprises of a number of perforation elements.

Another embodiment of the invention describes the device for the collecting of a blood sample according to the invention, in which the perforation system comprises at least 3 perforation elements.

An even further embodiment of the invention describes the device for the collection of a blood sample according to the invention, in which the holder comprises a second opening for receiving the sample object.

A fifth embodiment of the invention describes the device for the collection of a blood sample according to the invention, in which the holder comprises a designation, which allows for positioning the holder correctly in respect of the sample object.

Yet a further embodiment of the invention describes the device for the collection of a blood sample according to the invention, in which the holder comprises a recess that allows for checking the blood sample volume visually.

A further embodiment of the invention describes the device for the collection of a blood sample according to the invention, in which the container comprises an indicator for the indication of the required blood sample volume.

Another embodiment of the invention describes the device for collecting a blood sample according to the invention, in which the container allows for the storage of a blood sample.

A ninth embodiment of the invention describes the device for the collection of a blood sample according to the invention, the perforation system further comprising a sealing ring to seal a second opening of the container.

A further embodiment of the invention describes the device for the collection of a blood sample according to the invention, the casing further comprising a spring holder for attaching the spring and for attaching the perforation system in the casing.

Yet another embodiment of the invention describes the device for collecting a blood sample according to the invention, with the container further comprising a safety system that, after attachment of the container, prevents an activation of the perforation system and, after removing the safety system, facilitates the activation of the perforation system.

A further embodiment of the invention describes a kit, comprising the device for the collection of a blood sample and further comprising a ring for a temperature indication of the sample object.

A further embodiment of the invention describes the kit in accordance with the invention, further comprising a blood sample anticoagulant that is located on the inside of the container.

### SHORT DESCRIPTION OF THE FIGURES

Under specific reference to the figures, it is emphasized that the shown details only serve as manner of example and are only for the illustrative discussion of the different embodiments of this invention. They are proposed with the objective of the delivering of what can be seen as the most useful and immediate description of the principles and conceptual aspects of the invention. In this regard, no attempt is made to show more structural details of the invention as necessary for a fundamental understanding of the invention. The description in combination with the figures makes it clear for the experts in the field how the different forms of the invention can be implemented in practice.

FIG. 1A: Is a cross section of a medical device, according to an embodiment of the current invention.

FIG. 1B Is a medical device, according to an embodiment of the current invention.

FIG. 2: Is a detail recording of a medical device, according to an embodiment of the current invention.

FIG. 3: Is a cross section of a medical device, according to an embodiment of the current invention.

FIG. 4: Is a schematic rendition of a part of the kit of a medical device, according to an embodiment form of the current invention.

FIG. 5: Is a schematic rendition of a medical device, according to an embodiment of the current invention.

FIG. 6: Is a medical device, according to an embodiment of the current invention.

### DETAILLED DESCRIPTION OF THE INVENTION

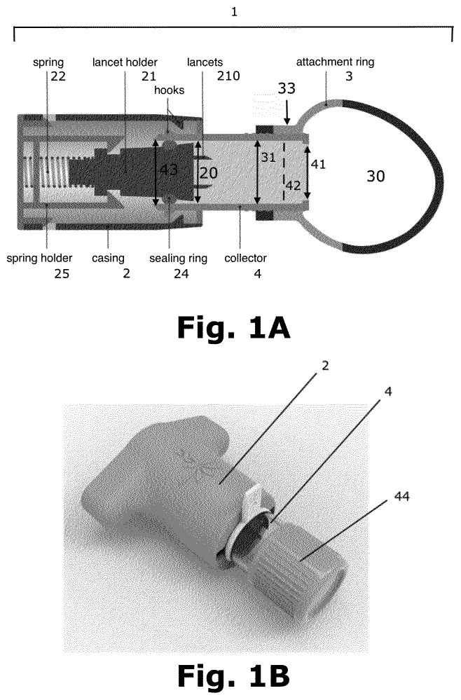

We refer to FIG. 1A and 1B, FIG. 1A shows a cross section of a medical device according to an embodiment of the current invention and FIG. 1B shows a medical device, according to an embodiment of the current invention. The collection phase starts with the placement of the attachment ring 3 (hereinafter also referred to as “holder”). The user preferably puts this on the ring finger of the non-dominant hand.

In addition, the holder 3 has a height difference 34. This prevents the remaining blood, after collection, from being smeared over the finger. A cut-out 33 (hereinafter also referred to as “recess”) is provided to facilitate quick monitoring of the quantity of blood.

In addition, a casing 2 and the container 4 (hereinafter also referred to as “collector”) can be used. An anticoagulant can also be used in the container 4 so that the collected blood sample does not clot. The user of the medical device 1 must first remove the cap 44 (hereinafter also referred to as “closing mechanism”) from the first opening 41 of the container 4 by means of a rotary motion. This causes a volume indicator 42 to appear (hereinafter also referred to as “indicator”). This is followed by turning the first opening 41 of the container 4 into the first opening 31 of the holder 3.

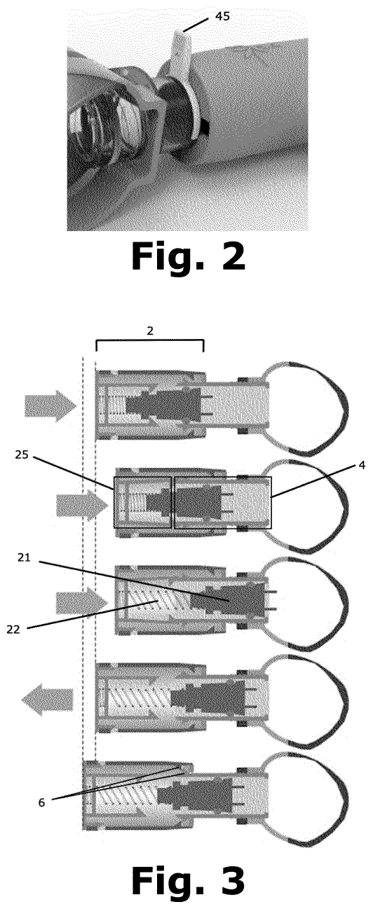

We refer to FIG. 2 that shows a detail recording of a medical device, according to an embodiment of the current invention. When fastening the container 4, there is the possibility of accidently discharging the lancets 210 (hereinafter also referred to as perforation elements). The security system 45 prevents that. By a simple pulling movement, the security system 45 can be removed from the outside of the container 4.

We refer to FIG. 3 that shows a series of cross sections of a medical device, according to an embodiment of the current invention. By pushing the casing 2 forward, the container 4 touches the spring holder 25, which subsequently opens and releases the perforation system (hereinafter also referred to as the “lancet holder”). The perforation system 21 moves forward quickly and punctures the finger when the kinetic energy is at its maximum. This is also the neutral position of the spring 22. The remaining energy, which is normally used for oscillation, is now fully used for puncturing. All of this ensures a quicker and accordingly less painful puncture.

The puncture is followed by the retraction. By stopping to push on the casing 2, it bounces back spontaneously through the legs of the spring holder 25. Finally, the user must briefly pull the casing 2 backward. This way, the system falls into a lock by means of the hooks 6 provided. To get a smoother communication of “how far” the pull must be, a lock will appear that shows the correct “locked” phase. This lock forms a fixed collection volume. A maximum volume of 750 μl can be collected. This gives sufficient volume for when something goes wrong.

After a successful collection, the casing 2 and the container are removed by a simple rotary movement. The closing mechanism 44 is subsequently tightened again and the holder 3 is removed.

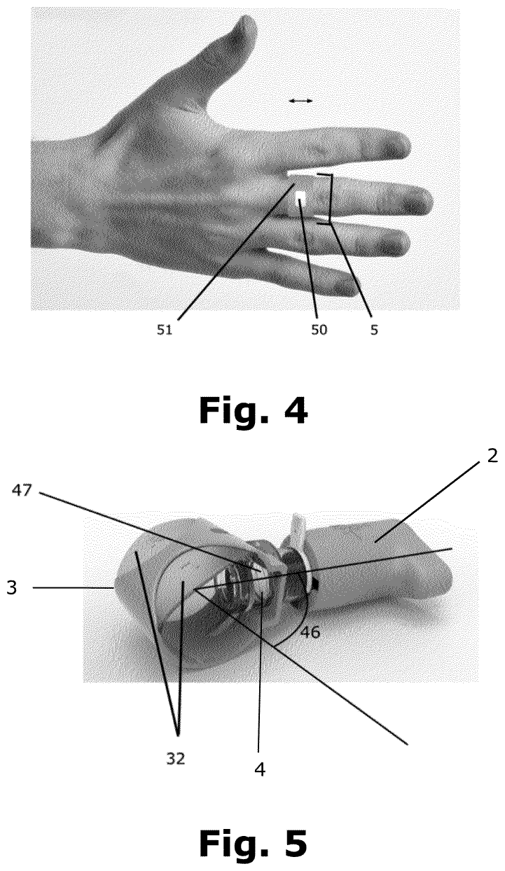

We refer to FIG. 4 showing a schematic rendition of a part of the kit of a medical device, according to an embodiment form of the current invention. The thermochromic ring 5 is a part of the kit of the current medical device, according to an embodiment form of the current invention, with which the user comes into contact. The ring is preferably put on the middle finger of the non-dominant hand. The user then lets warm water run. The user uses the ring as indicator for the heat of the water. This has a thermochromic zone 50 that changes colour between 42.5° C. and 45° C. Effective simulation can take place from when discolouration occurs. The user washes the hands for two minutes. The hands are then dried, and collection can begin.

Technically, the ring can be manufactured via co-injection of two silicones. One of the silicones 51 comprises the reference colour while the other silicone 50 has a thermochromic master batch. The ring approaches the form of the ring finger.

We refer to FIG. 5 that shows a schematic rendition of a medical device, according to an embodiment of the current invention.

By using a nail and right-left-designation 32 (hereinafter also referred to as “designation”), near the holder 3, people are still capable of finding the correct needle puncture site near the nail bed. Finally, the container 4 makes an angle of approximately 60 degrees 46 with the casing 2, which can be attached with the help of a double screw thread 47. Technically, the holder 3 can be manufactured by, via co-injection, a soft silicone and a hard-transparent polypropylene (hereinafter also referred to as “PP). The hard plastic ensures a smooth needle puncture collector attachment and a clear nail designation.

Components of the Invention

Below, we provide a summary of a number of useful materials with regard to different components of the invention. These materials are for illustration only and not limited to the current invention.

The first component or cap 44 can be an elementary screw-on cap with double screw thread. The unit can, for example, be die cast in PP. The cap 4 can be provided with ridges to accentuate the rotary motion.

The next component is the casing. The unit can be die cast in PP. The rear of component can be provided with ridges for additional grip.

The third component is preferably a transparent polycarbonate (hereinafter also referred to as PC) collector 4. The unit can be a die cast co-injection between the volume designation and the collector. The unit can be provided with a double screw thread. The correct amount of liquid EDTA can be added after assembly.

The fourth component or the safety system 45 can also be a die cast PC. The ridges and dart can ensure smooth removal.

The fifth component or the lancets 210 can be manufactured from stainless steel. As an example, we use a triple order lancet with a length of 2.4 mm and 0.64 mm diameter. This is analogous to the medical test. The lancet itself will preferably protrude approximately 3 mm from the lancet container 21.

Component six of the lancet holder 21 can be die cast in BP. We can attach the lancets with the help on the insert moulding. The unit can be made with a slope for a smooth installation of the seal. A hook can also be provided at the back for the attachment of the spring.

The spring 22 can be manufactured from spring steel. The spring has, for example 13 coils, a free length of 23 mm, a wire thickness of 0.5 mm and an outer diameter of 6 mm. These may still vary, depending on the actual application.

Finally, we have the spring holder 25. This component can also be die cast PP. Space can also be provided to attach the spring as well as two snap connectors. These connect the spring holder to the connector.

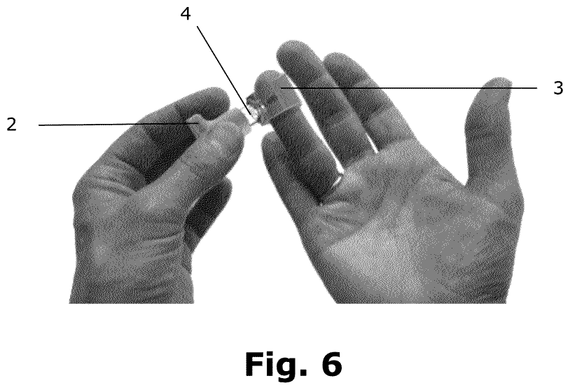

We refer to FIG. 6 that shows a medical device according to an embodiment of the current invention.

The holder 3 can be attached to the ring finger of the right hand. The container 4 can be tightened to the holder. The casing can be held with the help of the left hand to activate the medical device accordingly.

1-13. (canceled)

14. A device for collecting a blood sample, the device comprising:
a holder for receiving a sample object, wherein the holder defines an opening for attaching a container, the container making a direct connection between the holder and a casing, the container comprising a first opening and a closing mechanism for closing the first opening of the container;
wherein:
the casing comprises an opening, a perforation system, and a spring;
the perforation system is movable by the spring in the opening of the casing and in the container between a first position and a second position,
the perforation system is located at least partially outside the container in the second position;
the container further comprises a safety system that, after attachment of the container to the holder, prevents an activation of the perforation system and that, after removing the safety system, facilitates the activation the perforation system.

15. The device according to claim 14, wherein the perforation system comprises a number of perforation elements.

16. The device according to claim 15, wherein the perforation system comprises at least three perforation elements.

17. The device according to claim 16, wherein the holder comprises a second opening for receiving the sample object.

18. The device according to claim 14, wherein the holder comprises a designation that allows for correctly positioning the holder in respect of the sample object.

19. The device according to claim 14, wherein the holder comprises a recess that allows for visually checking the blood sample volume.

20. The device according to claim 14, wherein the holder comprises an indicator for the indication of the required blood sample volume.

21. The device according to claim 14, wherein the holder allows for storage of the blood sample.

22. The device according to claim 14, wherein the perforation system further comprises a sealing ring for closing the second opening of the container.

23. The device according to claim 14, wherein the casing further comprises a spring holder for the attachment of the spring and for the attachment of the perforation system in the housing.

24. A kit comprising the device according to claim 14 and a ring for a temperature indication of the sample object.

25. The kit according to claim 24, further comprising a blood sample anticoagulant located inside the container.
